(19) United States
(12) Patent Application Publication
(10) Pub. No.: US 2022/0338773 A1
(43) Pub. Date: Oct. 27, 2022

(54) METHOD AND SYSTEM TO EVALUATE CONCENTRATION OF A LIVING BEING

(71) Applicant: Panasonic Intellectual Property Management Co., Ltd., Osaka (JP)

(72) Inventors: Ai Min ZHAO, Singapore (SG); Faye Juliano, Singapore (SG); Eng Chye Lim, Singapore (SG); Nway Nway Aung, Singapore (SG); Souksakhone Bounyong, Nara (JP)

(21) Appl. No.: 17/696,561

(22) Filed: Mar. 16, 2022

Related U.S. Application Data
(60) Provisional application No. 63/180,016, filed on Apr. 26, 2021.

(51) Int. Cl. A61B 5/16; A61B 5/00; G06N 3/08; G06N 3/04

(52) U.S. Cl. CPC A61B 5/165; A61B 5/7246; A61B 5/7285; G06N 3/08; G06N 3/04

(57) ABSTRACT

The present subject matter refers a method to evaluate concentration of a living being based on artificial intelligent techniques. The method comprises detecting a continuous increase in concentration of a living being based on an artificial neural network (ANN), The method comprises receiving a parameter of the continuous increase in concentration, determining a first value of the concentration based on a first condition, said first condition defined by increase of the received parameter by more than a first predetermined threshold; and determining a second value of the concentration based on a second condition, said second condition defined by decline in the concentration from the first value by more than a second predetermined threshold.

### CROSS REFERENCE TO RELATED APPLICATION

The present application claims the benefit of U.S. Provisional Patent Application No. 63/180,016 filed on Apr. 26, 2021. The entire disclosures of the above-identified applications, including the specifications, drawings and claims are incorporated herein by reference in their entirety.

### TECHNICAL FIELD

The present invention generally relates to artificial-intelligence (AI) enabled inspection of processes and in particular, relates to detecting mental concentration.

### BACKGROUND

Solutions to optimize or personalize E-learning contents have been into existence for sometime. As may be understood, when living being learners are learning from e-learning device, their concentration levels are detected. The e-learning contents are adjusted based on the concentration level to suit each learner.

A japanese patent publication 2021-023492A refers concentration estimating part for estimating a degree of concentration of a subject based on data obtained from a sensor for monitoring a state of the subject. A centralized environment degree estimating part is provided for estimating a concentrated environmental level which is an index representing a degree of a highly concentrated environment based on data obtained from another sensor for monitoring a state around the subject.

Another japanese patent publication 2014-230717 refers a concentration level estimation device for estimating a concentration level of a subject. The device includes a heartbeat sensor for detecting the information on heartbeats from the subject, a concentration level estimation part for estimating a concentration level from the information on the heartbeats, a collection part for collecting a subjective concentration level correlated with the concentration level by the input of the subject, a reaction time, etc. A conversion part converts the subjective concentration level, the reaction time, etc. into a concentration level. A learning part learns the heartbeat information corresponding to the concentration level, the reaction time, etc, and the concentration level as learning data. The concentration level estimation part estimates the concentration level from the heartbeat information using the result of the learning from the learning part.

However, the state of the art concentration detection models output different absolute concentration levels depending on the training data or algorithm. So it remains extremely complex and tough to determine a threshold as to whether the learner is concentrating on the task or not.

There lies a need to evolve an intelligent concentration level detecting mechanism that determines the concentration based on real-time data. There lies a need of concentration level detecting mechanism that allows determination of the threshold related to the concentration with an ease of operation.

### SUMMARY

This summary is provided to introduce a selection of concepts in a simplified format that are further described in the detailed description of the present disclosure. This summary is not intended to identify key or essential inventive concepts of the claimed subject matter, nor is it intended for determining the scope of the claimed subject matter. In accordance with the purposes of the disclosure, the present disclosure as embodied and broadly described herein, describes method and system for predicting or classifying condition of any material or object or any feature/attribute related to the material/object either in a fully automated environment or through a semi-automatic mechanism.

The present subject matter refers a method to evaluate concentration of a living being based on artificial intelligent techniques. The method comprises detecting a continuous increase in concentration of a living being based on an artificial neural network (ANN). The method comprises receiving a parameter of the continuous increase in concentration, determining a first value of the concentration based on a first condition, said first condition defined by an increase of the received parameter by more than a first predetermined threshold; and determining a second value of the concentration based on a second condition, said second condition defined by a decline in the concentration from the first value by more than a second predetermined threshold.

The present subject matter recites a method with artificial intelligence (AI) technology for assessing attention span. The inventive method captures the video via webcam and predicts the concentration level with the extracted features from face, eye and head pose by an AI model that may be based on various sensors such as imaging sensor, acoustic sensor etc. The method analyzes the concentration level of a subject to detect a lap of attention and/or on the otherwise a lapse of attention as well. In an example, when a subject is concentrating on an eBook or a particular task, the concentration level exhibits rise and high that triggers a notable concentration event (NCE) as start of attention span. But when the subject loses attention from the eBook, the concentration level declines and a notable distract event (NDE) is detected as the end of concentration.

The objects and advantages of the embodiments will be realized and achieved at-least by the elements, features, and combinations particularly pointed out in the claims. It is to be understood that both the foregoing general description and the following detailed description are representative and explanatory and are not restrictive of the invention, as claimed.

### BRIEF DESCRIPTION OF THE DRAWINGS

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present invention. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

FIG. 1 illustrates method steps in accordance with the embodiment of the present subject matter;

FIG. 2 refers illustration of the method steps in accordance with an embodiment of the present subject matter;

FIGS. 3a and 3b illustrate example method steps and a scenario in accordance with an embodiment of the present subject matter;

FIG. 4 illustrates concentration detection in a scenario in accordance with an embodiment of the present subject matter;

FIG. 5 illustrates concentration detection in another scenario in accordance with an embodiment of the present subject matter;

FIG. 6 illustrates a view of GUI in accordance with an embodiment of the present subject matter;

FIG. 7 illustrates another view of GUI in accordance with an embodiment of the present subject matter;

FIG. 8 illustrates an implementation of the system as illustrated in preceding figures in a computing environment, in accordance with another embodiment of the present subject matter.

The elements in the drawings are illustrated for simplicity and may not have been necessarily been drawn to scale. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having the benefit of the description herein.

### DETAILED DESCRIPTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present invention. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skilled in the art to which this invention belongs. The system, methods, and examples provided herein are illustrative only and not intended to be limiting.

Embodiments of the present subject matter are described below in detail with reference to the accompanying drawings.

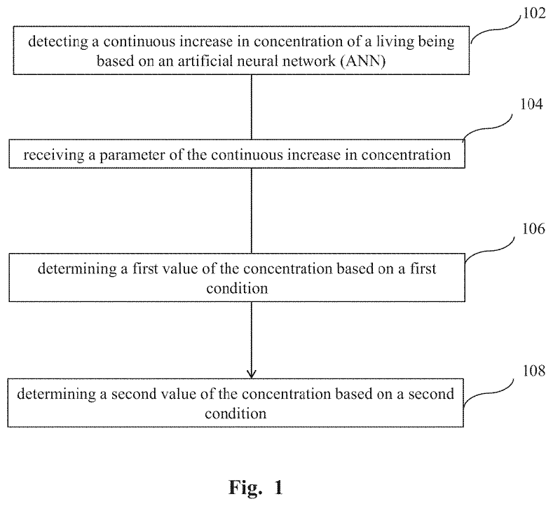

FIG. 1 illustrates method steps in accordance with an embodiment of the present subject matter. The method to evaluate concentration of a living being based on artificial intelligent techniques comprises detecting (step 102) a continuous increase in concentration of a living being based on an artificial neural network (ANN). The same is based on data captured from the information source, wherein the information source is at least one of an imaging device or acoustic device, the imaging device being an in-built or external camera.

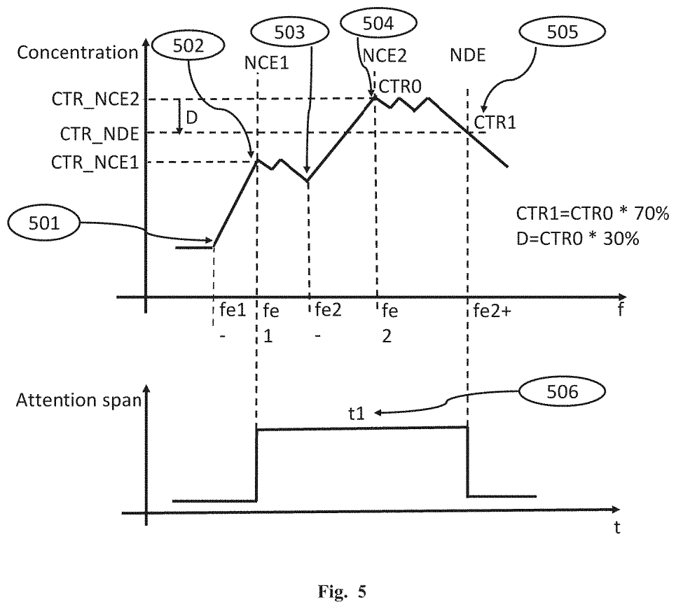

Further, the method comprises receiving (step 104) a parameter of the continuous increase in concentration and determining (step 106) a first value (NCE) of the concentration based on a first condition. The first value may be defined as a notable concentration event (NCE). The first condition is defined by an increase of the received parameter by more than a first predetermined threshold. The first condition is defined by a criteria whether a number of image frames captured during detecting the continuous increase of the concentration exceed the first threshold, the first threshold defining a number of frames configurable by a user. Further, the increase in concentration to a next higher value than the first value and complying the first condition results in an adjudication of the next higher value as a current first value that overrides earlier computed first value (as depicted in FIG. 5). However, as explained later, the attention span is calculated from the earlier most computed first value.

Further, the method comprises determining (step 108) a second value (NDE) of the concentration based on a second condition, wherein such second condition is defined by decline in the concentration from the first value (NCE) by more than a second predetermined threshold. The second value may be defined as a notable distract event (NDE). The second condition is defined by a criteria whether the second value is less than the current first value (or an initial first value in case of single first value) by the second predetermined threshold. The second threshold is a user-configurable percentage of the first value. In an example, unlike the first value which may be a set of values comprising the earlier first value and the current first value, the second value may be a single value.

Further, the method comprises outputting a waveform denoting a variation of concentration for a time-interval defined between the first value and the second value, and linking the outputted waveform with the user content. In other example, the waveform as outputted may be outputting a signal denoting an attention span for a time-interval defined between the first value and the second value. In case the first value is a set of the values (as depicted in FIG. 5), then time interval initiates from the initialmost first value, encompasses the later computed or the current first value, and ends at the second value.

Each attention span is defined by a particular time interval. There may be a set of attention spans separated by time gaps. Total duration of the set of attention spans is a sum of the particular time intervals associated with the attention spans within the set. In case of a single attention span, an overall concentration level is computed as a ratio of a) a duration of the attention span, and b) a total time period of observation. Thereafter, the concentration level is linked with user content.

In case of the existence of multiple attention spans or a set of attention spans, an overall concentration level is computed as a ratio of a) a total duration of the set of attention spans, and b) a total time period of observation. Thereafter, the overall concentration level is linked with user content.

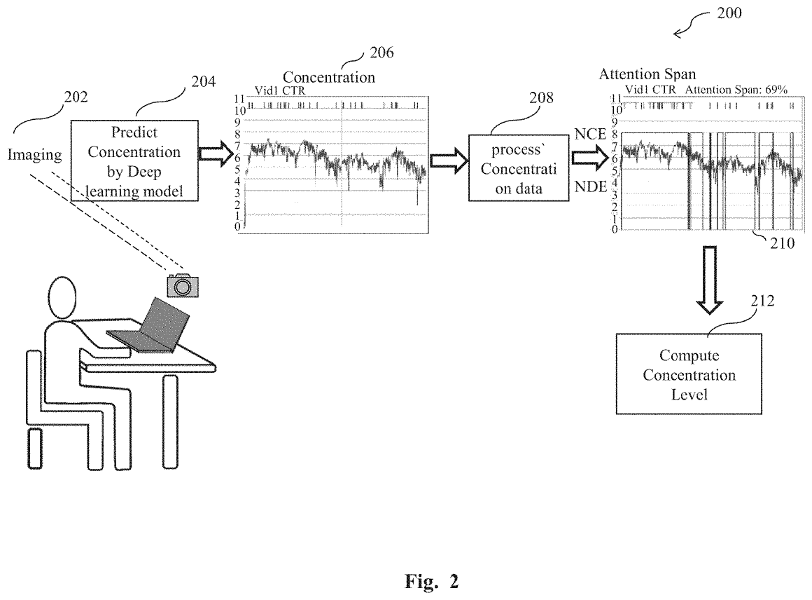

FIG. 2 refers illustration of the method steps in accordance with the present subject matter.

In accordance with the present subject matter, a personal device such as laptop may be used for measurement and detection of attention span, for example in the field of education.

Step 202 refers an example real-life scenario. While a living being or a subject is reading eBook or performing any particular task with such personal device such as the laptop, an application executing the background captures video data with an in-built or external camera associated with the personal device. In another example, the data as captured may be acoustic data or any other form of data capable of getting electronically captured. Step 202 corresponds to step 102 of FIG. 1.

Step 204 refers processing of video data or any other data in Step 202 by an algorithm in accordance with the present subject matter through a deep learning model or any other AI technique to identify and predict the concentration data of the subject under consideration. The present step 204 captures video via camera and predicts the concentration with the extracted features from face, eye and head pose through the AI model. Likewise, in case of acoustic data, the captured features may be acoustic features such as pitch, frequency, amplitude etc.

Step 206 refers an output of step 204 as a waveform or signal depicting the variation of concentration data as predicted by the AI model. Accordingly, the steps 204 till 206 collectively correspond to the step 104 of FIG. 1.

Step 208 refers monitoring changes of concentration data as captured in step 206 and accordingly identify within the concentration the occurrence of the NCE (Notable Concentration Event) and NDE (Notable Distract Event) in accordance with step 106 AND 108 of FIG. 1 that subsequently detect a lap of attention or an attention span.

When a subject is concentrating on an eBook or particular task, the concentration level exhibits rise and reaches a high that triggers the notable concentration event (NCE) as start of attention span. But when the subject loses attention to the eBook, the concentration level declines and Notable Distract Event (NDE) is detected as the end of attention span. NCE is triggered by consistent rise of concentration level for defined N frames (for example, 40 frames). As later referred in FIG. 5, CTR_NCE is a concentration level while NCE occurs. NDE is triggered by concentration decline to certain-level (for example, <CTR_NCE×70% as later referred in FIG. 5).

Step 210 refers an output of the step 208 wherein the waveform of step 206 is annotated with the attention spans (T1, T2, T3) as may be detected. As may be understood, T1 is an interval initiating from NCE and ending at next occurring NDE. Accordingly, a plurality of pairs of NCE and NDE lead to occurrence of T1, T2, T3 etc.

Step 212 denotes computation of concentration level as a ratio of attention span and time period of observation. For example, if the observation time is 1 hour within which three attention spans T1, T2, T3 occur, then the concentration level is calculated as:

(T1+T2+T3)/(1 hour).

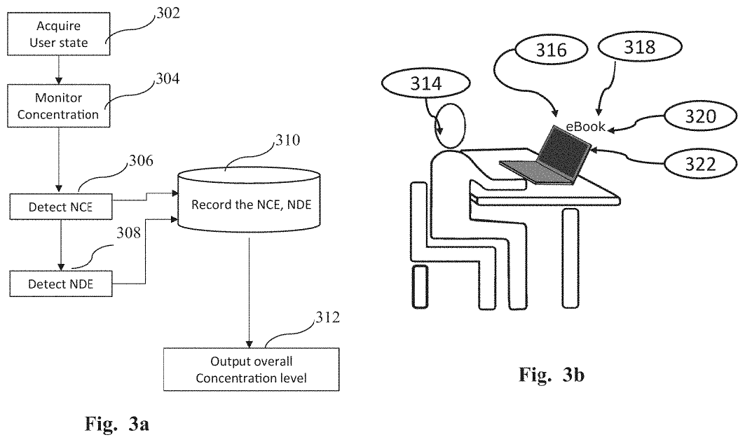

FIGS. 3a and 3b illustrate example method steps and a scenario in accordance with an embodiment of the present subject matter.

FIG. 3a illustrates method steps 302 till 312 to compute concentration level.

Step 302 acquires a user state of mind or focus in accordance with step 202.

Step 304 monitors concentration in accordance with the steps 204 and 206.

Step 306 and 308 recite detection of NCE and NDE in accordance with the step 208.

Step 310 refers logging of the concentration data as a waveform annotated with NCE and NDE in accordance with the step 210. Following table 1 depicts an example logging of attention spans or concentration span (CP) 1, 2 and 3 based on the pairs of NCE and NDE.

TABLE 1

| Concentration | NCE | NDE | E-book Link (Or link to |
|---|---|---|---|
| Span (CP) | (min) | (min) | distracted scene) |
| 1 | 2 | 7 | Link to CP1 |
| 2 | 10 | 12 | Link to CP2 |
| 3 | 15 | 20 | Link to CP3 |

Step 312 refers calculation of concentration level as the ratio in accordance with the step 212. The sum of concentration span in the present scenario may be defined as a sum of time durations associated with CP1, CP2 and CP3. In other example, each of CP1, CP2 and CP3 may be visualized as a flag having a time duration associated.

FIG. 3b illustrates measuring attention span according to the present subject matter while a student is reading eBook with a personal device such as a laptop.

Step 314 refers a student in classroom.

Step 316 refers a personal device, which could be a laptop, etc.

Step 318 refers a camera which may be in built camera or external webcam. In other example, instead of camera, an acoustic source such as a microphone may be provided.

Step 320 refers an eBook, other particular education program related entity, or anything that may be displayed and focused upon by a living being.

Step 322 refers a software application in accordance with steps 102 till 108 as executing in background within the computing system.

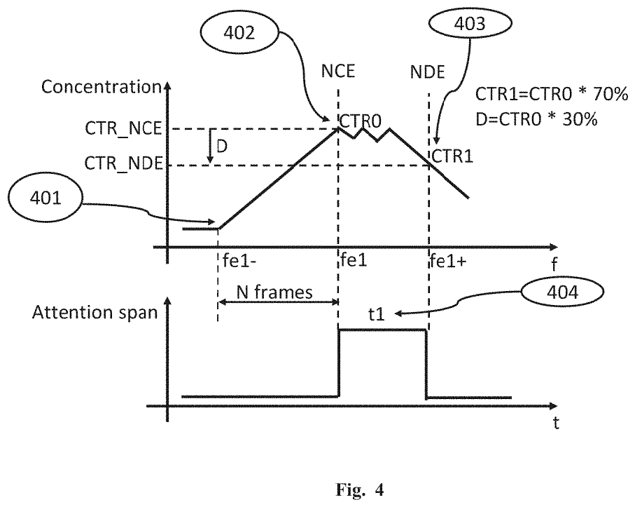

FIG. 4 illustrates NCE and NDE detection in an example scenario such that only one NCE is observed.

Step 401 refers a consistent rise of concentration and accordingly an initiation of the process in accordance with steps 102 and 104.

Step 402 refers detection of NCE if the concentration rise for more than N frames (i.e. compliance of the first condition). As may be understood, CTR0=CTR_NCE is recorded as reference for an Attention Span t1.

Step 403 refers detection of NDE if the concentration decline to CTR1=CTR_NDE<=CTR0*70% and thereby refer a compliance or meeting with the second condition.

Step 404 refers achievement of an Attention Span t1 waveform.

FIG. 5 refers NCE and NDE detection in scenario 2 wherein more than one NCE are observed.

Step 501 refers a consistent rise of concentration and accordingly an initiation of the present subject matter in accordance with steps 102 and 104.

Step 502 refers detection of NCE1 if the concentration rise for more than N frames (i.e. compliance of the first condition). As may be understood, CTR0=CTR_NCE1 is recorded as a reference of an Attention Span t1.

Step 503 refers a moment wherein concentration declines to a lower value. However, before NDE is detected, the concentration changes to a new consistent rise

Step 504 refers detection of NCE2 if the concentration rise for more than N frames. CTR0=CTR_NCE2 is recorded as new reference of Attention Span t1.

Step 505 refers detection of NDE if the concentration declines to CTR1=CTR_NDE<=CTR0*70%. In other words, NDE corresponds to a value that is 70% of NCE 2, even if NDE corresponds to a concentration larger than NCE1. Accordingly, NCE2 overrides the NCE 1 for the purposes of determination of NDE.

Steps 506 refers an Attention Span t1 waveform initiating from NCE1, encompassing NCE2 and concluding at NDE.

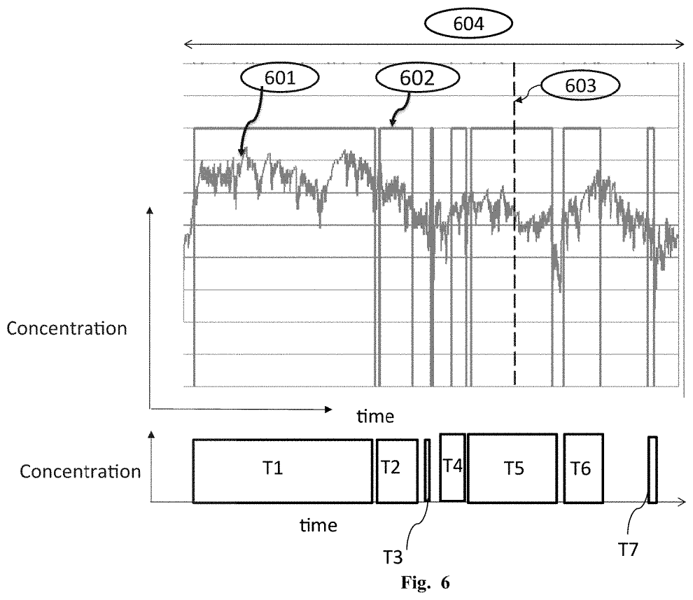

FIG. 6 illustrates a view of GUI showing attention Span detected according to the present subject matter. A first area of display-screen (not shown in figure) may be configured to receiving input data to display one or more user-controls for receiving a user input and thereby enable performance of the steps 102 till 108 of FIG. 1.

A second area of display-screen as shown in FIG. 6 may be configured to display the continuous increase in concentration, the first value and the second value of concentration; and. In an example, a GUI element 601 refers variation of concentration level data, a GUI element 602 refers one attention span T2 out of the multiple attention spans T1, T2, T3, T4, T5, T6 and T7. Accordingly, the GUI element 602 refers any of the multiple attention spans T1, T2, T3, T4, T5, T6 and T7.

GUI element 603 refers a user controllable indicator for navigating the first value (NCE1. NCE 2 etc), the second value (NDE) or any value located between the first and second value across any attention span. Such indicator may be provided on top for the detected NCE. In an example, each NCE indicator may be double-clicked to navigate or exhibit data in detail.

GUI element 604 refers computation of concentration level ratio against the whole video or time of observation in accordance with steps 212 and 312.

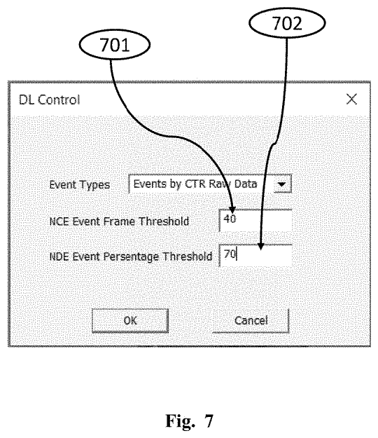

FIG. 7 illustrates another view of GUI showing the predefined parameters according to the present subject matter. More specifically, FIG. 7 refers a third area of display-screen configured to display a plurality of user-controls for configuring the first threshold (i.e. for NCE) and the second threshold (for NDE) associated with the first value and the second value of concentrations. The third area for configuring the threshold provides a dialog for configuring a time duration based parameter associated with the increment of the concentration to reach the first value.

The dialog may be used for configuring a number of frames required to be captured during the increment of the concentration to reach the first value. GUI element 701 refers a predefined parameter for NCE threshold which may be for example set as “40 frames”.

The dialog may be used for configuring a percentage of decline of the concentration to reach the second value of concentration from the first value of concentration. GUI element 702 refers predefined parameter for NDE threshold which may be for example set as “70%”.

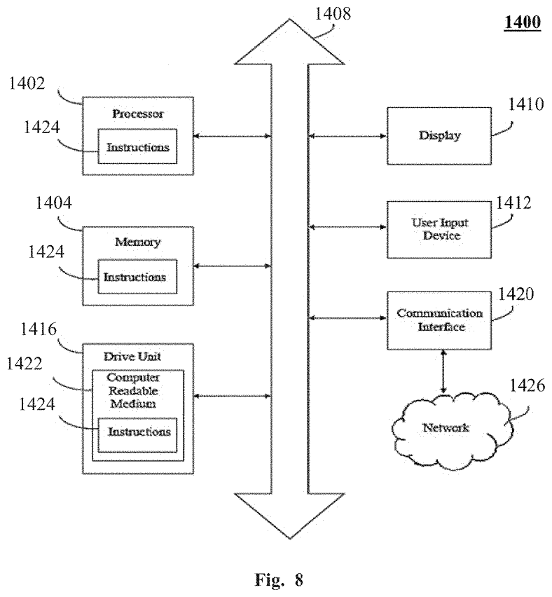

FIG. 8 illustrates an implementation of the system as illustrated in FIG. 1 till 2 in a computing environment. The present figure essentially illustrates the hardware configuration of the system. The computer system 1400 can include a set of instructions that can be executed to cause the computer system 1400 to perform any one or more of the methods disclosed. The computer system 1400 may operate as a standalone device or may be connected, e.g., using a network, to other computer systems or peripheral devices.

In a networked deployment, the computer system 1400 may operate in the capacity of a server or as a client user computer in a server-client user network environment, or as a peer computer system in a peer-to-peer (or distributed) network environment. The computer system 1400 can also be implemented as or incorporated into various devices, such as a personal computer (PC), a tablet PC, a personal digital assistant (PDA), a mobile device, a palmtop computer, a laptop computer, a desktop computer, a communications device, a wireless telephone, a land-line telephone, a web appliance, a network router, switch or bridge, or any other machine capable of executing a set of instructions (sequential or otherwise) that specify actions to be taken by that machine. Further, while a single computer system 1400 is illustrated, the term “system” shall also be taken to include any collection of systems or sub-systems that individually or jointly execute a set, or multiple sets, of instructions to perform one or more computer functions.

The computer system 1400 may include a processor 1402 (e.g., a central processing unit (CPU), a graphics processing unit (GPU), or both. The processor 1402 may be a component in a variety of systems. For example, the processor 1402 may be part of a standard personal computer or a workstation. The processor 1402 may be one or more general processors, digital signal processors, application specific integrated circuits, field programmable gate arrays, servers, networks, digital circuits, analog circuits, combinations thereof, or other now known or later developed devices for analyzing and processing data The processor 1402 may implement a software program, such as code generated manually (i.e., programmed).

The computer system 1400 may include a memory 1404, such as a memory 1404 that can communicate via a bus 1408. The memory 1404 may be a main memory, a static memory, or a dynamic memory. The memory 1404 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one example, the memory 1404 includes a cache or random access memory for the processor 1402. In alternative examples, the memory 1404 is separate from the processor 1402, such as a cache memory of a processor, the system memory, or other memory. The memory 1404 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 1404 is operable to store instructions executable by the processor 1402. The functions, acts or tasks illustrated in the figures or described may be performed by the programmed processor 1402 executing the instructions stored in the memory 1404. The functions, acts or tasks are independent of the particular type of instructions set, storage media, processor or processing strategy and may be performed by software, hardware, integrated circuits, firm-ware, micro-code and the like, operating alone or in combination. Likewise, processing strategies may include multiprocessing, multitasking, parallel processing and the like.

As shown, the computer system 1400 may or may not further include a display unit 1410, such as a liquid crystal display (LCD), an organic light emitting diode (OLED), a flat panel display, a solid state display, a cathode ray tube (CRT), a projector, a printer or other now known or later developed display device for outputting determined information. The display 1410 may act as an interface for the user to see the functioning of the processor 1402, or specifically as an interface with the software stored in the memory 1404 or in the drive unit 1416.

Additionally, the computer system 1400 may include an input device 1412 configured to allow a user to interact with any of the components of system 1400. The input device 1412 may be a number pad, a keyboard, or a cursor control device, such as a mouse, or a joystick, touch screen display, remote control or any other device operative to interact with the computer system 1400.

The computer system 1400 may also include a disk or optical drive unit 1416. The disk drive unit 1416 may include a computer-readable medium 1422 in which one or more sets of instructions 1424, e.g. software, can be embedded. Further, the instructions 1424 may embody one or more of the methods or logic as described. In a particular example, the instructions 1424 may reside completely, or at least partially, within the memory 1404 or within the processor 1402 during execution by the computer system 1400. The memory 1404 and the processor 1402 also may include computer-readable media as discussed above.

The present invention contemplates a computer-readable medium that includes instructions 1424 or receives and executes instructions 1424 responsive to a propagated signal so that a device connected to a network 1426 can communicate voice, video, audio, images or any other data over the network 1426. Further, the instructions 1424 may be transmitted or received over the network 1426 via a communication port or interface 1420 or using a bus 1408. The communication port or interface 1420 may be a part of the processor 1402 or may be a separate component. The communication port 1420 may be created in software or may be a physical connection in hardware. The communication port 1420 may be configured to connect with a network 1426, external media, the display 1410, or any other components in system 1400 or combinations thereof. The connection with the network 1426 may be a physical connection, such as a wired Ethernet connection or may be established wirelessly as discussed later. Likewise, the additional connections with other components of the system 1400 may be physical connections or may be established wirelessly. The network 1426 may alternatively be directly connected to the bus 1408.

The network 1426 may include wired networks, wireless networks, Ethernet AVB networks, or combinations thereof. The wireless network may be a cellular telephone network, an 802.11, 802.16, 802.20, 802.1Q or WiMax network. Further, the network 1426 may be a public network, such as the Internet, a private network, such as an intranet, or combinations thereof, and may utilize a variety of networking protocols now available or later developed including, but not limited to TCP/IP based networking protocols.

In an alternative example, dedicated hardware implementations, such as application specific integrated circuits, programmable logic arrays and other hardware devices, can be constructed to implement various parts of the system 1400.

The present subject matter at least provides monitoring changes of concentration which trigger NCE (Notable Concentration Event) and NDE (Notable Distract Event) that subsequently detect the laps of attention and lapses in concentration. The present subject matter corresponds to a generic concentration detection model that may be used across a wide variety of applications requiring concentration determination. The concentration data is predicted by AI technology and based thereupon concentration data waveform is used for detection of attention span. Through the GUI, the present subject matter provides a tuning parameter for triggering notable concentration event and the same is rendered configurable. Overall, the configurability of thresholds allows measurement of attention span with more accuracy. In an example, the statistic results of attention span may be used in education as the feedback for a teacher.

Terms used in this disclosure and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description of embodiments, claims, or drawings, should be understood to contemplate the possibilities of including one of the terms, either of the terms or both terms. For example, the phrase “A or B” should be understood to include the possibilities of “A” or “B” or “A and B.”

All examples and conditional language recited in this disclosure are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that various changes, substitutions, and alterations could be made thereto without departing from the spirit and scope of the present disclosure.

We claim:

1. A method to evaluate concentration of a living being based on artificial intelligent techniques comprising:
detecting a continuous increase in concentration of a living being based on an artificial neural network (ANN);
receiving a parameter of the continuous increase in concentration;
determining a first value of the concentration based on a first condition, said first condition defined by increase of the received parameter by more than a first predetermined threshold; and
determining a second value of the concentration based on a second condition, said second condition defined by decline in the concentration from the first value by more than a second predetermined threshold.

2. The method to evaluate concentration according to claim 1, further comprising:
outputting a waveform denoting a variation of concentration for a time-interval defined between the first value and the second value; and
optionally linking the outputted waveform with the user content.

3. The method to evaluate concentration according to claim 1, further comprising:
outputting a signal denoting an attention span for a time-interval defined between the first value and the second value; and
optionally linking the outputted signal with the user content.

4. The method to evaluate concentration according to claim 3, further comprising:
obtaining a set of attention spans, each attention span defined by a particular time interval; and
linking the set of attention spans with the user content, wherein a total duration of the set of attention spans is a sum of the particular time intervals associated with the attention spans within the set.

5. The method to evaluate concentration according to claim 4, further comprising:
computing an overall concentration level as a ratio of a) a total duration of the set of attention spans, and b) a total time-period of observation; and
linking the overall concentration level with a user content.

6. The method to evaluate concentration according to claim 1, wherein the first condition is defined by a criteria whether a number of image frames captured during detecting the continuous increase of the concentration exceed the first threshold, the first threshold defining a number of frames configurable by a user.

7. The method to evaluate concentration according to claim 1, wherein the second condition is defined by a criteria whether the second value is less than the first value by the second predetermined threshold.

8. The method to evaluate concentration according to claim 5, wherein the second threshold is a user configurable percentage of the first value.

9. A system to evaluate concentration of a living being based on artificial intelligent techniques comprising:
an information source;
an artificial neural network (ANN) for detecting a continuous increase in concentration of a living being based on data captured from the information source;
a computing unit configured for the steps of:
receiving a parameter of the continuous increase in concentration;
determining a first value of the concentration based on a first condition, said first condition defined by increase of the received parameter by more than a first predetermined threshold; and
determining a second value of the concentration based on a second condition, said second condition defined by decline in the concentration from the first value by more than a second predetermined threshold.

10. The system as claimed in claim 9, wherein the information source is at least one of an imaging device or acoustic device, the imaging device being an in-built or external camera.

11. The system as claimed in claim 9, wherein the computing unit is further configured for:
outputting a signal denoting an attention span for a time-interval defined between the first value and the second value; and
optionally linking the outputted signal with the user content.

12. The system as claimed in claim 11, wherein the computing unit is further configured for:
obtaining a set of attention spans, each attention span defined by a particular time interval; and
linking the set of attention spans with the user content, wherein a total duration of the set of attention spans is a sum of the particular time intervals associated with the attention spans within the set.

13. The system as claimed in claim 12, further comprising:
computing an overall concentration level as a ratio of a) a total duration of the set of attention spans, and b) a total time-period of observation; and
linking the overall concentration level with a user content.

14. A Graphical user interface (GUI) to evaluate concentration of a living being based on artificial intelligent techniques, said GUI comprising:
a first area of display-screen configured to receiving input data to display one or more user-controls for receiving a user input and thereby enable performance of the steps of:
detecting a continuous increase in concentration of a living being based on an artificial neural network (ANN);
receiving a parameter of the continuous increase in concentration;
determining a first value of the concentration based on a first condition, said first condition defined by increase of the received parameter by more than a first threshold; and
determining a second value of the concentration based on a second condition, said second condition defined by decline in the concentration from the first value by more than a second threshold.
a second area of display-screen configured to display the continuous increase in concentration, the first value and the second value of concentration; and
a third area of display-screen configured to display a plurality of user-controls for configuring the first threshold and the second threshold associated with the first value and the second value of concentrations.

15. The GUI as claimed in claim 14, wherein the third area for configuring the threshold provides a dialog for configuring one or more of:
a time duration based parameter associated with the increment of the concentration to reach the first value;
a number of frames required to be captured during the increment of the concentration to reach the first value; and
a percentage of decline of the concentration to reach the second value of concentration from the first value of concentration.

16. The GUI as claimed in claim 14, further comprising a user controllable indicator for navigating one or more of:
the first value;
the second value; and
any value located between the first value and the second value within an attention span defined as an interval between the first value and the second value.

17. The GUI as claimed in claim 14, wherein the first area is further configured for:
computing an overall concentration level as a ratio of a) a total duration of the set of attention spans, and b) a total time-period of observation; and
linking the overall concentration level with a user content.

18. The GUI as claimed in claim 14, wherein the first condition is defined by a criteria whether a number of image frames captured during detecting the continuous increase of the concentration exceed the first threshold, the first threshold defining a number of frames configurable by a user.

19. The GUI as claimed in claim 14, wherein the second condition is defined by a criteria whether the second value is less than the first value by the second predetermined threshold.

20. The GUI as claimed in claim 14, wherein the second threshold is a user configurable percentage of the first value.
